SEQUENCE LISTING (8 sequences)

SEQ ID NO: 1 - DNA, 19 bp, Artificial Sequence
  Description of Artificial Sequence Synthetic primer
gggaagctgttgtcccaag

SEQ ID NO: 2 - DNA, 21 bp, Artificial Sequence
  Description of Artificial Sequence Synthetic primer
caaggggaggtaaaagagagc

SEQ ID NO: 3 - DNA, 19 bp, Artificial Sequence
  Description of Artificial Sequence Synthetic primer
agccacatcgctcagacac

SEQ ID NO: 4 - DNA, 19 bp, Artificial Sequence
  Description of Artificial Sequence Synthetic primer
gcccaatacgaccaaatcc

SEQ ID NO: 5 - DNA, 20 bp, Artificial Sequence
  Description of Artificial Sequence Synthetic primer
cgctcagggtcacaagaaac

SEQ ID NO: 6 - DNA, 20 bp, Artificial Sequence
  Description of Artificial Sequence Synthetic primer
gaggcaaggaggaaaacaca

SEQ ID NO: 7 - DNA, 20 bp, Artificial Sequence
  Description of Artificial Sequence Synthetic primer
ttccatccagttgccttctt

SEQ ID NO: 8 - DNA, 20 bp, Artificial Sequence
  Description of Artificial Sequence Synthetic primer
atttccacgatttcccagag

(19) United States
(12) Patent Application Publication
(10) Pub. No.: US 2022/0218652 A1
(43) Pub. Date: Jul. 14, 2022

(54) METHODS OF TREATING CYTOKINE RELEASE SYNDROME

(71) Applicants: The General Hospital Corporation, Boston, MA (US); AZTherapies, Inc., Boston, MA (US)

(72) Inventors: David R. Elmaleh, Newton, MA (US); Rudolph E. Tanzi, Milton, MA (US); Ana Griciuc, Arlington, MA (US); Robert Warren, Boston, MA (US); Karen Reeves, Boston, MA (US)

(21) Appl. No.: 17/273,646

(22) PCT Filed: Sep. 5, 2019
(86) PCT No.: PCT/US2019/049733

§ 371 Date: Mar. 4, 2021

Related U.S. Application Data
(60) Provisional application No. 62/727,177, filed on Sep. 5, 2018.

(51) Int. Cl. A61K 31/352; A61P 37/06; A61K 45/06; A61K 38/17

(52) U.S. Cl. CPC A61K 31/352; A61K 38/1709; A61K 45/06; A61P 37/06

(57) ABSTRACT

The present disclosure relates to a method of treating at least one Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution Inflammatory Syndrome (IRIS), Graft-Versus-Host Disease (GVHD), Acute Respiratory Distress Syndrome (ARDS), sepsis, Ebola, avian influenza, smallpox, Systemic Inflammatory Response Syndrome (SIRS), and Immune-related Adverse Events Syndrome (IrAES) in a subject in need thereof, comprising administering a mast cell stabilizer or a compound of Formula I or Formula II: Formula I, Formula II, wherein R¹ is halogen, OH, or —OC(O)C₁₋₅alkyl R² and R³ are each independently selected from CO₂R⁴ or CH²OR⁵; R⁴ is i L, Na, K, H, C₁₋₅alkyl, or —CH₂CO(C₁₋₅alkyl); and R⁵ is H or C(O)(C₁₋₅alkyl), or a pharmaceutically acceptable salt thereof.

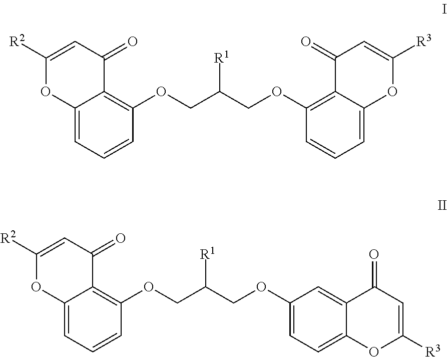

### CROSS-REFERENCE TO RELATED APPLICATIONS

This application is the U,S. National Stage of International Patent Application No. PCT/U52019/049733, filed Sep. 5, 2019, which claims priority to U.S. Provisional Patent Application No. 62/727,177, filed Sep. 5, 2018, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Nov. 16, 2021, is named MAA-03001_SL.txt and is 2.1 kilobytes in size.

### BACKGROUND

Immune-based biologics are targeted therapies that are impacting the treatment of cancer and other diseases. These treatments are more effective than chemotherapy for several tumor types, as well as autoimmune and inflammatory diseases, However, these treatments, in most cases, are associated with complex, toxicity-related side effects known as Drug-Related. Adverse Events (DRAEs). The treatments induce systemic reactions that activate or inhibit cellular messaging signals, causing immunosuppression, cell hyperactivity, and cell destruction. These side effects or syndromes have been referred to by different clinical names such as: Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution inflammatory Syndrome (IRIS), graft-versus-host disease (GYM), systemic inflammatory response syndrome (SIRS), and Immune-related Adverse Events Syndrome (IrAES). Some of these conditions refer to the observed side effects during or following treatments with biologics. These syndromes can be categorized by their associated mode of administration or mechanism of action. Unfortunately, some of these side effects are severe and may be fatal. An adjunctive treatment is required to lower systemic toxicity.

Biomedicine drugs or biologics are drugs produced by living organisms, such as cloned proteins, products of recombinant DNA, DNA gene therapies, biomanufacturing products, and synthetic drug preparations made from nucleotides or amino acids. Some common biologics are monoclonal antibodies (mAbs) and their fragments, peptides, fusion proteins, and vaccines. This growing class of therapeutics includes compounds for treating various indications in oncology, genetic diseases, and autoimmune diseases, Most biologics are associated with adverse events from chronic or route of administration, i.e., inhalation, intravenous (IV), subcutaneous (SQ) and intramuscular (IM) injection. Drug administration depends on many factors such as molecular size, physical properties of these biologics (such as lipophilicity and gastric degradation that prevent the biologics from gastric. absorption). In some cases, dry powder and aerosol formulation have been approved for some biologics.

Biologics, such as bispecific T-cell engaging (BiTE) single-chain antibody constructs and Immune Effector Cells (TECs), including T cells and natural killer cells, which are genetically engineered to express a chimeric antigen receptor adaptive T cells (CAR-T), alone or in combination with chemotherapy and radiation, are part of the most modem armament for fighting specific cancers. These treatments exhibit great efficacy. Unfortunately, they are associated with toxic side effects and immunogenicity from their infusion and treatment, In most cases the systemic toxicity is treated and can be overcome. Sometimes, however, the side effects are severe and require extensive emergent treatment. CRS and ICANS are among potential side effects of treatment with biologics; symptoms of CRS and ICANS may appear immediately or hours following infusion. In some instances, these treatment modalities can affect the brain causing a cancer-related cognitive impairment, also known as “chemo brain”.

Studies of CAR-T treatment indicate that toxicity is associated with the appearance of many pro-inflammatory cytokines, including interleukin IL-1, IL-13, soluble IL-6 receptor, soluble interferon gamma (INFγ), and tumor necrosis factor alpha (TNFα). CAR-T-mAb, such as muromonab-CD₃, anti-CD52 (alemtuzumab), anti-CD20 (rituximab), and the CD:28 super-agonist, theralizumab, cause B cell, T cell (lymphocytes), macrophage, dendritic cell, and monocytes (myeloid) activation and the release of pro-inflammatory cytokines. Therefore, it is important to develop drugs that transform these activated cells into an anti-inflammatory state (e.g., phagocytic macrophages that remove cytokines and toxins) that will reduce cytokine release and relieve the. severity of the CRS and ICANS symptoms.

### SUMMARY OF THE INVENTION

The present disclosure relates to a method of treating at least one condition selected from Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution Inflammatory Syndrome (IRIS), Graft-Versus-Host Disease (GVHD), Acute Respiratory Distress Syndrome (ARDS), sepsis, Ebola, avian influenza, smallpox, Systemic Inflammatory Response Syndrome (SIRS), and Immune-related Adverse Events Syndrome (IrAES) in a subject in need thereof, comprising administering a compound of Formula I or Formula ii:

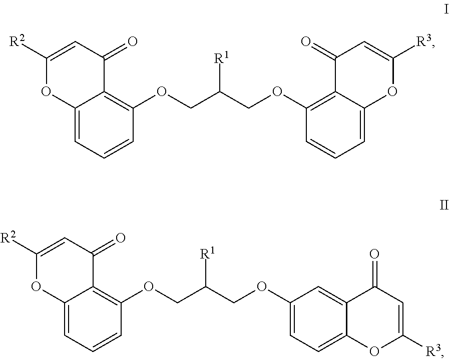

wherein

R¹ is halogen, OH, or —OC(O)C₁₋₅alkyl

R² and R³ are each independently selected from CO₂R⁴ or CH₂OR⁵;

R⁴ is Li, Na, K, H, C₁₋₅alkyl, or —CH₂CO(C₁₋₅alkyl); and

R⁵ is H or C(O)(C₁₋₅alkyl),

or a pharmaceutically acceptable salt thereof.

The present disclosure also relates to a method of treating at least one condition selected from Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic inflammatory Response Syndrome (SIRS), Immune Reconstitution inflammatory Syndrome (IRIS), graft-versus-host disease (GVHD), acute respiratory distress syndrome (ARDS), sepsis, Ebola, avian influenza, smallpox, systemic inflammatory response syndrome (SIRS), and Immune-related Adverse Events Syndrome (IrAES) in a subject in need thereof, comprising administering a mast cell stabilizer.

The present disclosure also relates to a method of treating at least one condition selected from Cytokine Release Syndrome (CRS), Immune effector Cell-Associated Neurotoxicity Syndrome (ICANS), cancer-related cognitive impairment, infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), immune Reconstitution Inflammatory Syndrome (IRIS), graft-versus-host disease (GVHD), acute respiratory distress syndrome (ARDS), sepsis, Ebola, avian influenza, smallpox, systemic inflammatory response syndrome (SIRS), and Immune-related Adverse Events Syndrome (frAES) in a subject in need thereof, comprising administering a compound selected from the compounds of Formula III, Formula IV, Formula V, Formula VI, Formula VII, Formula VIII, Formula IX, Formula X, Formula XI, Formula XII, Formula XIII, and Formula XIV:

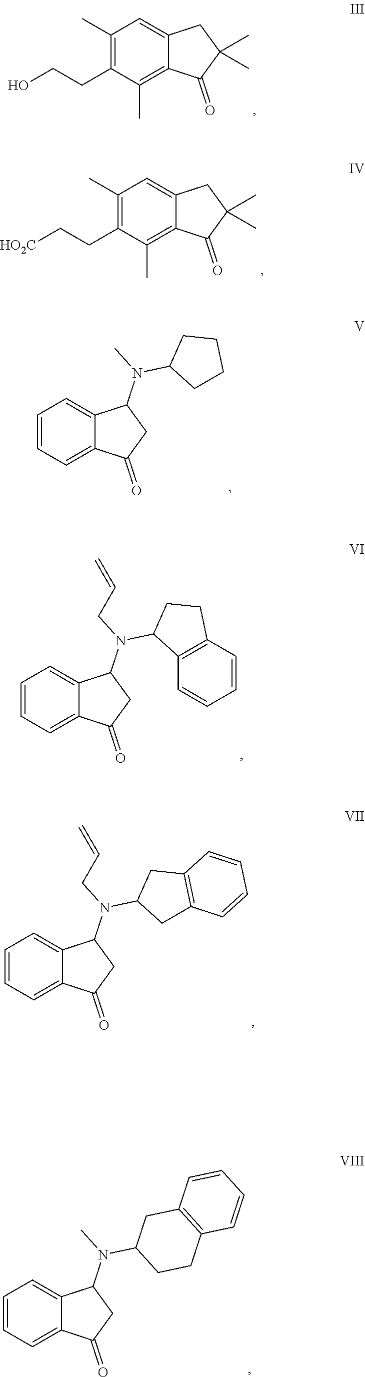 [SMILES: Cc1cc2c(c(C)c1CCO)C(=O)C(C)(C)C2 ; Cc1cc2c(c(C)c1CCC(=O)O)C(=O)C(C)(C)C2 ; CN(C1CCCC1)C1CC(=O)c2ccccc21 ; C=CCN(C1CCc2ccccc21)C1CC(=O)c2ccccc21 ; C=CCN(C1Cc2ccccc2C1)C1CC(=O)c2ccccc21 ; CN(C1CCc2ccccc2C1)C1CC(=O)c2ccccc21]

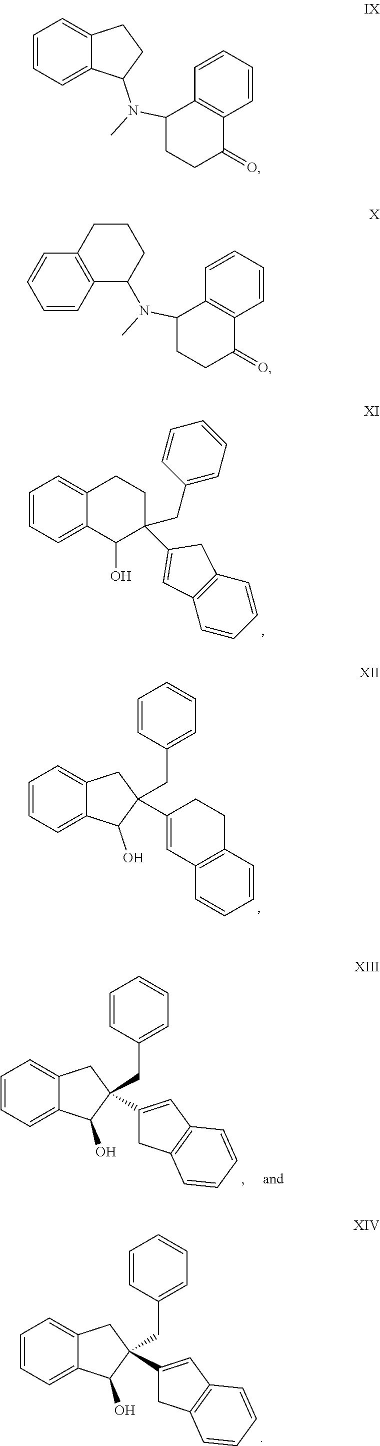 [SMILES: CN(C1CCc2ccccc21)C1CCC(=O)c2ccccc21 ; CN(C1CCCc2ccccc21)C1CCC(=O)c2ccccc21 ; OC1c2ccccc2CCC1(Cc1ccccc1)C1=Cc2ccccc2C1 ; OC1c2ccccc2CC1(Cc1ccccc1)C1=Cc2ccccc2CC1 ; O[C@H]1c2ccccc2C[C@]1(Cc1ccccc1)C1=Cc2ccccc2C1 ; O[C@H]1c2ccccc2C[C@@]1(Cc1ccccc1)C1=Cc2ccccc2C1]

The present disclosure also relates to a method of treating at least one condition selected from Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), immune Reconstitution Inflammatory Syndrome (IRIS), graft-versus-host disease (GVHD), acute respiratory distress syndrome (ARDS), sepsis, Ebola, avian influenza, smallpox, systemic inflammatory response syndrome (SIRS), and Immune-related Adverse Events Syndrome (IrAES) in a subject in need thereof, comprising administering an anti-inflammatory small molecular peptide truncated from anti-inflammatory gene protein.

### BRIEF DESCRIPTION OF THE DRAWINGS

FIGS. 1A-1D are graphs showing that cromolyn treatment decreased the levels of pro-inflammatory cytokines in the spinal cord of TgSOD1 mice. FIG. 1A: IL-1β. FIG. 1B: IL-5. FIG. 1C: IL-6. FIG. 1D: TNFα. * denotes differences between TgSOD1-Vehicle and Tg-SOD1-Cromolyn; \hat{ } denotes differences between TgSOD1-Vehicle and WtSOD1-Vehicle; # denotes differences between TgSOD1-Vehicle and WtSOD1-Cromolyn; @ denotes differences between TgSOD1-Cromolyn and WiSOD1-Vehilce; % denotes differences between TgSOD1-Cromolyn and WtSOD1-Cromolyn. *p<0.05; **p<0.01; ***p<0.001; ***p<0.0001, the same statistical significance is associated with each symbol. Data are presented as median and interquartile ranges.

FIGS. 2A-2F are graphs showing that cromolyn treatment decreased the levels of pro-inflammatory cytokines in plasma of TgSOD1 mice. FIG. 2A: IL-1β. FIG. 2B: IL-2. FIG. 2C: IL-5. FIG. 2D: IL-6. FIG. 2E: IL-10, FIG. 2F: TNFα. * denotes differences between TgSOD1-Vehicle and Tg-SOD1-Cromolyn; A denotes differences between TgSOD1-Vehicle and WtSOD1-Vehicle; # denotes differences between TgSOD1-Vehicle and WtSOD1-Cromolyn; @ denotes differences between TgSOD1-Cromolyn and WtSOD1-Vehilce; % denotes differences between TgSOD1-Cromolyn and WtSOD1-Cromolyn, *p<0.05; **p<0.01; ***p<0.001; ***p<0.0001, the same statistical sianificance is associated with each symbol. Data are presented as median and interquartile ranges.

FIGS. 3A-3F are images and graphs demonstrating that cromolyn reverses pro-inflammatory CD33-mediated inhibition of M1-microglial activation stage in APP/PS1 mice. 3A-3D: confocal microaraphs of BV2 microglial cells treated with fluorescently-labeled Aβ42 (red), plasma membrane dye (PM, green), and either DMSO (control) or cromolyn sodium in DMSO. 3A: DMSO+PM+Aβ42. 3B: DMSO+Aβ42. 3C: cromolyn sodium+PM+Aβ42. 3D: cromolyn sodium+Aβ42. 3E: ELISA analysis of Aβ42 uptake by the BV2 microglial cells treated with different concentrations of cromolyn sodium. 3F: ELISA analysis of Aβ42 uptake by the BV2-CD33^{WT} microglial cells treated with different concentrations of cromolyn sodium.

FIGS. 4A-4B are graphs demonstrating gene expression of IL-1β (FIG. 4A) and IL-6 (FIG. 4B) in N9 microglia cell line stimulated with LPS and treated with different concentrations of cromolyn.

### DETAILED DESCRIPTION OF THE INVENTION

### Overview

Following treatment with a biologics, inflammation and immune changes can exacerbate the damage or play a protective role, depending on types of cytokines and cells involved in the interactions. The protective aspects of inflammation include clearance of debris by microglia in the brain, which is important in repair and interaction with T cells. It is known that the changes in properties of microglia, the brain-resident macrophages, depend on their response to different stimuli in their microenvironment (e.g., cytokines), resulting in a range of phenotypes. Based on the changes in expression of cytokines, receptors, and other markers, monocyte and macrophage states have been defined as following: classical activation (M1), alternative activation (M2a), type II alternative activation (M2b), and acquired deactivation (M2c).

M1 activated microglia can produce reactive oxygen species and result in increased production of pro-inflammatory cytokines such as TNFα and IL-1.

Macrophage M2 activation is associated with mediators that are known to contribute to the anti-inflammatory actions and reorganization of extracellular matrix. Microglia with M2a phenotypes have increased phagocytosis and produce growth factors such as insulin-like growth factor-1 and anti-inflammatory cytokines such as IL-10. Stimulation of macrophages by IL-4 and/or IL-13 results in an M2a state, sometimes called a wound-healing macrophage and it is generally characterized by low production of pro-inflammatory cytokines (IL-1, TNF and IL-6). IL-4 is known to be an important modulator of M2a microglial activation. The M2a responses are primarily observed in allergic responses, extracellular matrix deposition, and remodeling.

M2b macrophages are unique in that they express high levels of pro-inflammatory cytokines, characteristic of M1 activation, but also express high levels of the anti-inflammatory cytokine IL-10.

Finally, the M2c macrophage state is stimulated by IL-10 and is sometimes referred to as a regulatory macrophage. M2c macrophages have anti-inflammatory activity that plays a role in the phagocytosis of cellular debris without the classical pro-inflammatory response. These cells express transforming growth factor-β (TGF-β) and high IL-10 as well as matrix proteins. IL-10 mediates anti-inflammatory responses including decreasing glial activation and production of pro-inflammatory cytokines.

Two avenues of study have been pursued over the years: research into anti-inflammatory agents to temper toxic effect of pro-inflammatory cytokines; and studies focused on converting microglia from this M1 state to an M2 state, in which the toxic effects are reduced and their phagocytic activity is enhanced. It is generally accepted that activation of monocytes and microglia has potential to decelerate neurodegenerative progression by modulating immune responses to increase the intrinsic phagocytic capacity of monocytes and microglia without triggering secretion of pro-inflammatory cytokines that could worsen neurodegeneration. Recent studies demonstrate that cromolyn exhibits significant M2 microglial activation, a phagocytic stage of damage repair by brain microglia.

The present invention relates in part to a series of compounds, including cromolyn and its derivatives, and their combinations to serve as adjuvant drugs for attenuating the cytokine release associated with administration of biologics. In some embodiments, the drugs are designed to protect patients undergoing treatment with biologics. In certain embodiments, the adjuvant drugs are applied as pre-treatment, as part of treatment and/or post-treatment. in some embodiments, the adjuvant drugs attenuate the pro-inflammatory toxicity associated with the administration of certain biologics. In some embodiments, where there is no adjuvant drug-biotherapeutic agent interaction, the biologics and the adjuvant drug are co-administered. In some embodiments, the proposed adjuvant drug could be co-administered by the same delivery mode e., as infusion) as the biologics, or by other modes (i.e., infusion+IP or Sub Q). In some embodiments, CRS is dampened due to effective phagocytic microglia activation promoted by the adjuvant drugs, enabling removal of cytokines and toxins that were induced by biologics administration. In certain embodiments, specific targeted drugs, such as monoclonal antibodies, CAR-T cell therapy, gene therapy, miRNA, siRNA, CRISPR or their combinations as CRS inducers are subject to the adjuvant drug treatment. In some embodiments, the adjuvant drugs are used with targeted brain cancer treatment associated with cancer-related cognitive impairment, also known as “chemo brain” toxicity. in some embodiments, adjuvant therapy in many fashions to attenuate or prevent systemic toxicity.

The present invention relates in part to a family of adjuvant drugs that decrease or eliminate the harmful effects of cancer and immune-regulatory molecular treatments, including monoclonal antibodies, CAR-T cell therapy, gene therapy, miRNA, siRNA and future developed CRISPR drugs. In some embodiments the adjuvant drugs may work alone or in combination with other drugs to reduce or eliminate the effects of CRS after biologic drug therapy.

In some embodiments the adjuvant drugs could be combined with other immune suppressant drugs or other drugs that reduce CRS, such as corticosteroids, dopamine, norepinephrine, and etanercept.

In certain embodiments these drugs act specifically to prevent, reduce or eliminate the symptoms of cancer-related cognitive impairment, also known as “chemo brain”. In certain embodiments, these syndromes are reduced or eliminated with or without corticosteroids, dopamine and norepinephrine, etanercept.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cantbridae Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

When any variable (e.g., aryl, heterocyclyl, R², R^{a}, etc.) occurs more than once in a compound, its definition on each occurrence is independent of any other occurrence.

Unless specifically stated or obvious from context, as used herein, the term “or” is understood to be inclusive. Unless specifically stated or obvious from context, as used herein, the terms “a”, “an”, and “the” are understood to be singular or plural.

An “alkyl” group or “alkane” is a straight chained or branched non-aromatic hydrocarbon which is completely saturated. Typically, a straight chained or branched alkyl group has from I to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, formyl, or an acyl), is thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamide, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), −CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as alkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as tritluoromethyl and 2,2,2-tirfluoroethyl, etc.

The terms “halo” and “halogen” as used herein meanshalogen and includes chloro, fluoro, bromo, and iodo.

The term “substituted” refers to moieties haying substituents replacing a hydrogen on one or more carbons of the backbone. It will be understood that “substitution” or “substituted with” includes the implicit proviso that such substitution is in accordance with permitted valence of the. substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyciic and heterocyclic, aromatic and non-aromatic substituents of organic compounds. The permissible substituents can he one or more and the same. or different for appropriate organic compounds. it will be understood by those skilled in the art that substituents can themselves be substituted, if appropriate. Unless specifically stated as “unsubstituted,” references to chemical moieties herein are understood to include substituted variants. For example, reference to an “alkyl” group or moiety implicitly includes both substituted and unsubstituted variants.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include acetate, benzenesuifonate, benzoate, bicarbonate, bitartrate, bromide, calcium edetate, carasylate, carbonate, chloride, citrate, dihydrochloride, edetate, edisylate, estolate, esylate, fumarate, glyceptate, gluconate, glutamate, glycoilylarsanilate, hexyiresorcinate, hydrobromide, hydrochloride, hydroxynaphthoate, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teociate, tosylate, and triethiodide salts.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylatnine, morpholine, pyridine, piperidine, picoline, dicyclohexylamine, N,N′-dibenzyleilaylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylarnine, glucamine, N-methyigiucamine, collidine, quinine, quinoline, and basic amino acid such as lysine and arginine.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite skies of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When the geometry of a disclosed compound is named or depicted by structure, the named or depicted geometrical isomer is at least 60%, 70%, 80%, 90%, 99% or 99.9% by weight pure relative to the other geometrical isomers.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and: or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of ne or more symptoms of the disorder or condition relative to the untreated control sample.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition.

As used herein, the term “biologic” refers to a pharmaceutical drug product manufactured in, extracted from, or semisynthesized from biological sources. Biologics are isolated from a variety of natural sources—human, animal, microorganism, fungus, or plant, or they can be produced by recombinant DNA. Biologics include, but are not limited to, vaccines, whole blood, blood components, allergenics, somatic cells, gene therapies, tissues, organ transplants, cloned proteins, products of recombinant DNA, DNA gene therapies, miRNA, siRNA, drug preparations comprising nucleotides or amino acids, monoclonal antibodies (mAbs) and their fragments, peptides, fusion proteins, recombinant therapeutic proteins, glycoproteins, and living cells used in cell therapy. For example, the term “biologics” refers to hormones, such as insulin, erythropoietin, or growth-stimulating hormone, to monoclonal antibodies (mAb), or to receptor constructs such as fusion proteins. Additionally, the term “biologics” refers to immunotherapy agents, including IECs such as lymphocytes, macrophages, dendritic cells, natural killer cells, cytotoxic T lymphocytes (CTL), and CAR-T cells.

As used herein, the term “cell therapy” refers to therapy in which cellular material is injected, grafted or implanted into a patient to help lessen or cure a disease. Cell therapy involves transfer of living cells. The cells may originate from the patient (autologous cells) or a donor (allogeneic cells). Cell therapy can refer to therapy involving transfer of hematopoietic stem cells, CAR-T cells, other genetically modified T cells, vaccines, and natural killer cells.

As used herein, the term “gene therapy” refers to the therapeutic delivery of nucleic acid into a patient's cells as a drug to treat disease, Gene therapy can be used to reduce levels of a disease-causing version of a protein, increase production of disease-fighting proteins, or to produce new/modified proteins. Gene therapy includes several types of gene modifications: gene addition, gene correction, gene silencing, reprogramming, and cell elimination.

A “therapeutically effective amount”, as used herein refers to an amount that is sufficient to achieve a desired therapeutic effect. For example, a therapeutically effective amount can refer to an amount that is sufficient to improve at least one sign or symptom of diseases or conditions disclosed herein.

### Pharmaceutical Compositions

In certain embodiments, the present invention provides a pharmaceutical composition, comprising a compound of Formula I, Formula II, Formula III, Formula. IV, Formula V, Formula VI, Formula VII, Formula VIII, Formula IX, Formula X, Formula XI, Formula XII, Formula XIII, or Formula XIV, a mast cell stabilizer, or an anti-inflammatory small molecular peptide truncated from anti-inflammatory gene protein, and a pharmaceutically acceptable excipient.

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also he present in a transdermal delivery system, e.g., a skin patch. The composition can also he present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can he a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt: (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar, (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eve drop). The compound may also be for for inhalation. in certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, the contents of which are incorporated herein by reference in their entirety, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the subject being treated, the particular mode of administration. The amount of active inaredient that. can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium/phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) hurnectants, such as glycerol; (4) disintegrating agents, such as agar-agar, calcium carbonate, potato or tapioca. starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, tnay contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition an active compound, excipients, such as animal and vegetable fats, oils, waxes, paraffins, starch,tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide, powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference in their entirety. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile, isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming tnicroencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemuisions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocotnpatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administrotion, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. (Isselbacher et aL (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylarnino)ethanol, ethanolamine, ethylenediamine, N-methylglucamine, hydrabamine, 1H-imidazole lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryi sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble. antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treatment

In certain embodiments, the compounds of the invention and pharmaceutically acceptable salts or solvates thereof are administered in combination with a therapeutically effective amount of another therapeutic agent.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body or while the side effects of the previously administered therapeutic compound are still evident in the body (e. g. , the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of Formula I or II) or the one or more additional therapeutic a.gent(s). In certain such embodiments, the conjoint administration provides an additive effect, wherein an additive effect refers to the sum of each of the effects of individual administration of the compound of the invention and the one or more additional therapeutic agent(s). In other embodiments, the conjoint administration of a compound of the invention reduces or ameliorates the side effects of the additional therapeutic agent.

The therapeutic agent may be administered simultaneously with the compound of the invention. Alternatively, the therapeutic agent may be administered prior to administration the compound of the invention. Alternatively still, the therapeutic agent may be administered following the administration of the compound of the invention.

The phrase “combination therapy” embraces the administration of the compound of Formula I and an additional therapeutic agent as part of a specific treatment regimen intended to provide a beneficial effect from the co-action of each. When administered as a combination, the oligodendrocyte precursor differentiation inducing compound (the compound of Formula I) and an additional therapeutic agent can be formulated as separate compositions. Administration of these therapeutic agents in combination typically is carried out over a defined time period (usually minutes, hours, days or weeks depending upon the combination selected).

“Combination therapy” is intended to embrace administration of these therapeutic agent. (the compound of Formula I or Formula II and an additional therapeutic agent) in a sequential manner, that is, wherein each therapeutic agent is administered at a different time, as well as administration of these therapeutic agents, or at least two of the therapeutic agents, in a substantially simultaneous manner Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be. administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence wherein the therapeutic agents are administered is not narrowly critical. “Combination therapy” also can embrace the administration of the therapeutic agents as described above in further combination with other biologically active ingredients (such as, but not limited to, a second and different therapeutic agent) and non-drug therapies (e.g., surgery).

Administration methods include administering an effective amount of a compound or composition of the invention at different times during the course of therapy or concurrently in a combination form. The methods of the invention include all known therapeutic treatment regimens. In certain embodiments, the compound or pharmaceutical composition is administered intravenously, intrathecally, subcutaneously, intramuscularly, intranasally, or orally.

In certain embodiments, the compound of the invention is administered as an Ha salt. “Metabolite,” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 0.01 mg/kg/day to about 1000 mg/kg/day, from about 0.1 mg/kg/day to about 100 mg/kg/day, from about 0.5 mg/kg/day to about 50 mg/kg/day, or from about 1 mg/kg/day to 10 mg/kg/day.

“Pharmaceutically acceptable carrier” means compounds and compositions that are of sufficient purity and quality for use in the formulation of a composition of the invention and that, when appropriately administered to an animal or human, do not produce an adverse reaction.

### Methods of Preparation.

Compounds of the invention may be prepared according to the synthetic procedures described below. In cases where the synthetic intermediates and final products of Formula I described below contain potentially reactive functional groups, for example amino, hydroxy, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not usually described explicitly. Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCl in reaction mixtures are generally used in slight to substantial excess (1.05-5 equivalents).

Compounds of the invention can be prepared employing conventional methods that utilize readily available reagents and starting materials. The reagents used in the preparation of the compounds of this invention can be either commercially obtained or can be prepared by standard procedures described in the literature. The compounds of the invention may he made according to the general and exemplary schemes provided herein.

### Exemplary Compositions and Methods

The present disclosure relates to a method of treating or preventing at least one condition, wherein the condition is selected from Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution Inflammatory Syndrome (IRIS), Graft-Versus-Host. Disease (GVHD), Acute Respiratory Distress Syndrome (ARDS), sepsis, Ebola, avian influenza, smallpox, Systemic Inflammatory Response Syndrome (SIRS), and Immune-related Adverse Events Syndrome (IrAES) in a subject in need thereof, comprising administering a compound of Formula I or Formula II:

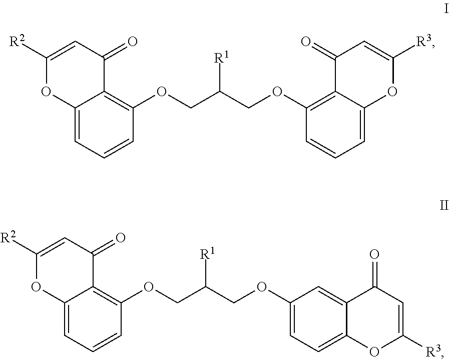

wherein

R¹ is halogen, OH, or —OC(O)C₁₋₅alkyl

R² and R³ are each independently selected from CO₂R⁴ or CH₂OR⁵;

R⁴ is Li, Na, K, H, C₁₋₅alkyl, or —CH₂CO(C₁₋₅alkyl); and

R⁵ is H or C(O)(C₁₋₅alkyl),

or a pharmaceutically acceptable salt thereof.

In some embodiments, R¹ is halogen, for example, R¹ is F. In certain embodiments, R¹ is OH. In some embodiments, R¹ is —OC(O)C₁₋₄alkyl, such as —OC(O)Me.

In certain embodiments, R² and R³ is each independently —CO₂R⁴. In some embodiments, R⁴ is Li, Na, K, or NH₄, for example, R⁴ is Na. In certain embodiments, R⁴ is H. In some embodiments, R⁴ is C₁₋₅alkyl. In certain embodiments, R⁴ is —CH₂CO(C₁₋₅alkyl);

In certain embodiments, R² and R³ is each independently —CH₂OR⁵. In some embodiments, R⁵ is H. In certain embodiments, R⁵ is —C(O)(C₁₋₅alkyl).

In some embodiments, C₁₋₅alkyl is methyl, ethyl, or t-butyl.

In certain embodiments, the compound of Formula I is selected from:

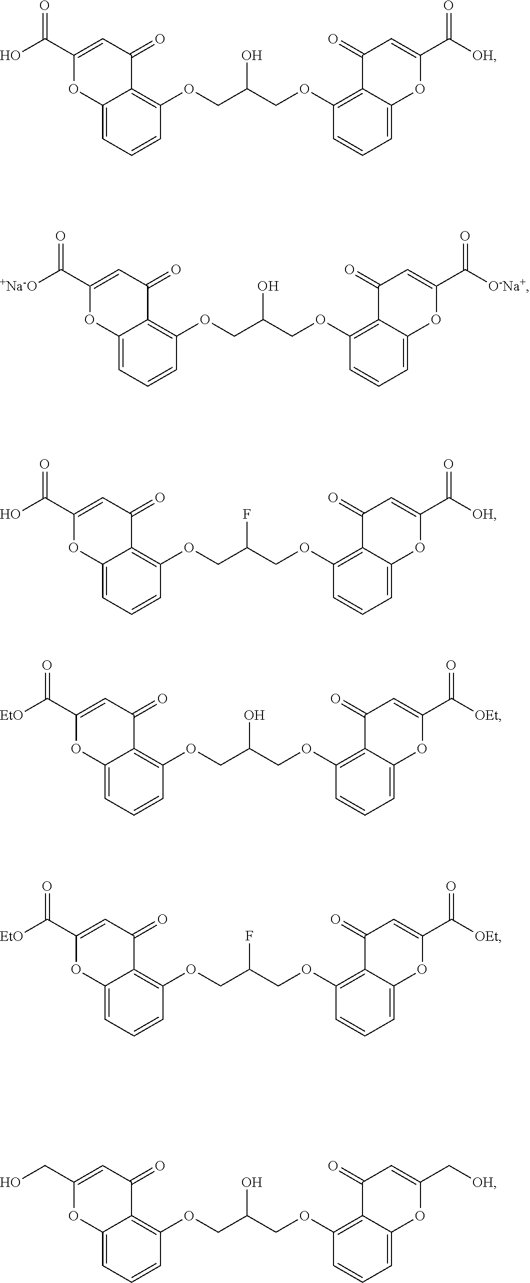

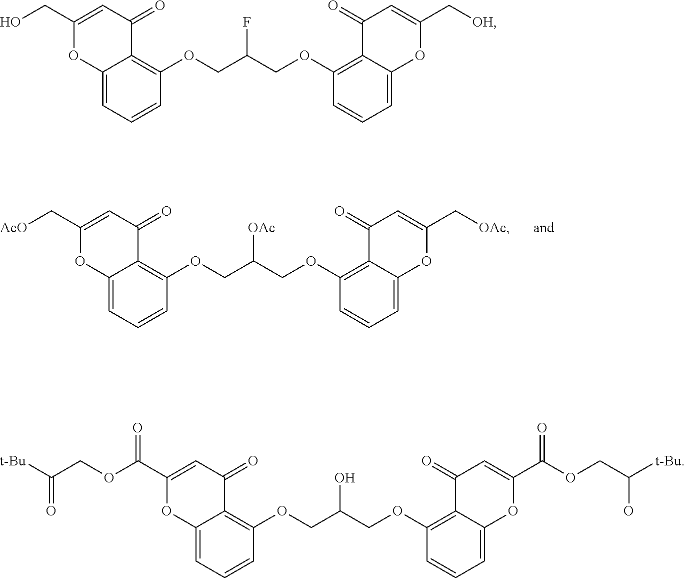 [SMILES: O=c1cc(CO)oc2cccc(OCC(F)COc3cccc4oc(CO)cc(=O)c34)c12 ; CC(=O)OCc1cc(=O)c2c(OCC(COc3cccc4oc(COC(C)=O)cc(=O)c34)OC(C)=O)cccc2o1 ; CC(C)(C)C(=O)COC(=O)c1cc(=O)c2c(OCC(O)COc3cccc4oc(C(=O)OCC(O)C(C)(C)C)cc(=O)c34)cccc2o1]

In certain embodiments, the compound of Formula I is

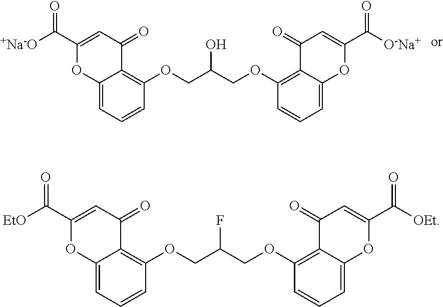

In certain embodiments, the compound of Formula I is

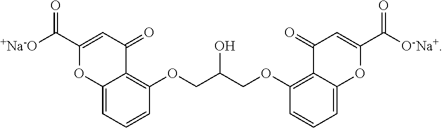

In certain embodiments, the compound of Formula I is

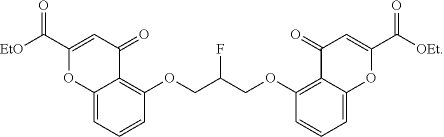 [SMILES: CCOC(=O)c1cc(=O)c2c(OCC(F)COc3cccc4oc(C(=O)OCC)cc(=O)c34)cccc2o1]

In certain embodiments, the condition is IrAES, cancer-related cognitive impairment, CRS, or ICANS; and the compound of Formula I is

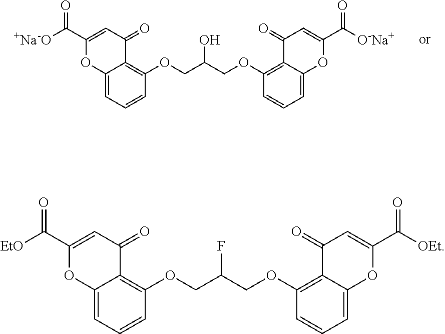

For example, the condition is CRS; and the compound of Formula I is

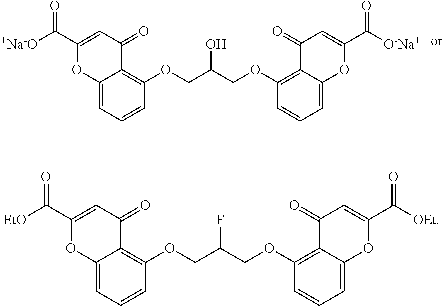

Alternatively, the condition is ICANS; and the compound of Formula I is

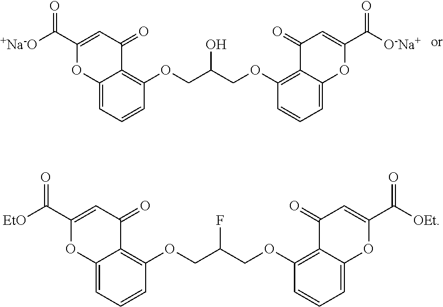

In some embodiments, the condition is cancer-related cognitive impairment; and the compound of Formula I is

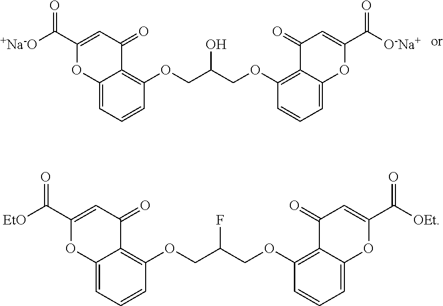

The present disclosure also relates to a method of treating or preventing at least one condition selected from Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), immune Reconstitution Inflammatory Syndrome (IRIS), graft-versus-host disease (GVHD), acute respiratory distress syndrome (ARDS), sepsis, Ebola, avian influenza, smallpox, systemic inflammatory response syndrome (SIRS), and Immune-related Adverse Events Syndrome (IrAES) in a subject in need thereof, comprising administering a mast cell stabilizer.

In some embodiments the mast cell stabilizer is selected from nedocromil, ketotifen, quercetin, omalizumab, olopatadine, azelastine, niepolizumab, methyl xanthines, and β2-adrenergic agonists.

The present disclosure also relates to a method of treating or preventing at least one condition selected from Cytokirte Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution Inflammatory Syndrome (IRIS), graft-versus-host disease (GVHD), acute respiratory distress syndrome (ARDS), sepsis, Ebola, avian influenza, smallpox, systemic inflammatory response syndrome (SIRS), and Immune-related Adverse Events Syndrome (IrAES) in a subject in need thereof, comprising administering a compound selected from the compounds of Formula III, Formula IV, Formula V, Formula VI, Formula VII, Formula VIII, Formula IX, Formula X, Formula XI, Formula XII, Formula XIII, and Formula XIV:

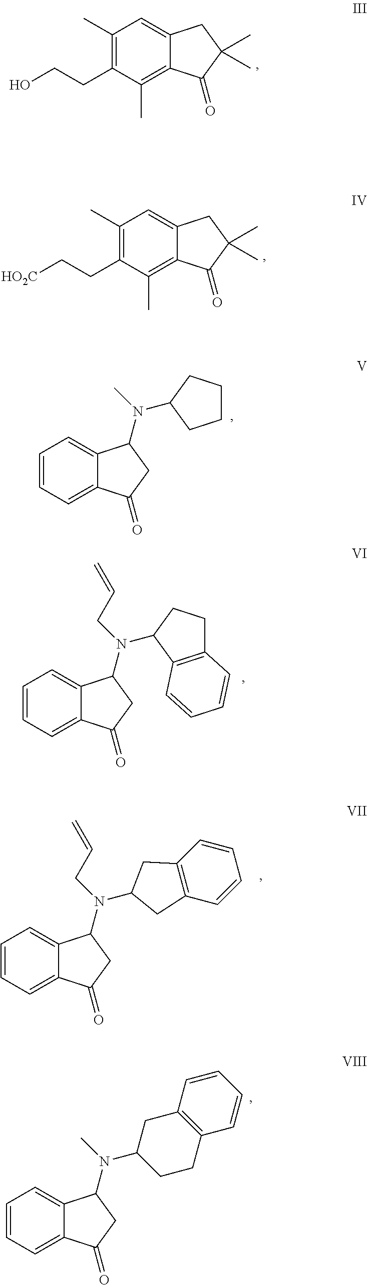 [SMILES: Cc1cc2c(c(C)c1CCO)C(=O)C(C)(C)C2 ; Cc1cc2c(c(C)c1CCC(=O)O)C(=O)C(C)(C)C2 ; CN(C1CCCC1)C1CC(=O)c2ccccc21 ; C=CCN(C1CCc2ccccc21)C1CC(=O)c2ccccc21 ; C=CCN(C1Cc2ccccc2C1)C1CC(=O)c2ccccc21 ; CN(C1CCc2ccccc2C1)C1CC(=O)c2ccccc21]

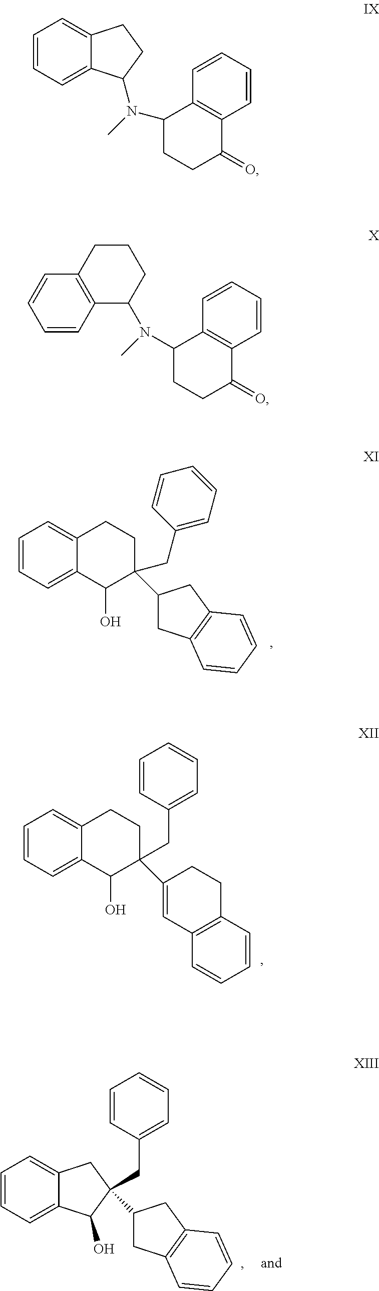

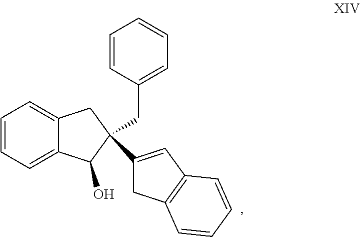 [SMILES: O[C@H]1c2ccccc2C[C@@]1(Cc1ccccc1)C1=Cc2ccccc2C1]

The present disclosure also relates to a method of treating or preventing at least one condition selected from Cytokine Release Syndrome (CRS), Immune effector Cell-Associated Neurotoxicitv Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution inflammatory Syndrome (IRIS), graft-versus-host disease (GVHD), acute respiratory distress syndrome (ARDS), sepsis, Ebola, avian influenza, smallpox, systemic inflammatory response syndrome (SIRS), and Immune-related Adverse Events Syndrome (IrAES) in a subject in need thereof, comprising administering an anti-inflammatory small molecular peptide truncated from anti-inflammatory gene protein. In some embodiments the anti-inflammatory gene protein is TREM2.

In certain embodiments, the condition is IrAES, cancer-related cognitive impairment, CRS, or ICANS. For example, the condition is CRS. Alternatively, the condition is ICANS. In some embodiments, the condition is cancer-related cognitive impairment.

In certain embodiments, the method further comprises administering one or more biologics. In some embodiments, the biologics is selected from vaccines, whole blood, blood components, allergenics, somatic cells, gene therapies, tissues, organ transplants, cloned proteins, products of recombinant DNA, DNA gene therapies, miRNA, siRNA, drug preparations comprising nucleotides or amino acids, monoclonal antibodies (mAbs), mAb fragments, peptides, fusion proteins, recombinant therapeutic proteins, glycoproteins, and living cells used in cell therapy. For example, the biologics is selected from vaccines, somatic cells, gene therapies, monoclonal antibodies (mAbs), mAb fragments, and living cells used in cell therapy. In some embodiments, the biologics is selected from hi-specific T-cell engagers, single-chain antibody constructs, and immune effector cells, such as CAR-T cells. Alternatively, the biologics is selected from IFN-γ, TNFα, muromonab-CD₃, alemtuzurnab, rituximab, solitomab, theralizumab, and blinatumomab.

In certain embodiments, the biologics is conjointly administered with the compound of Formula I or Formula II. For example, wherein the biologics, is administered prior to the compound of Formula I or Formula II. Alternatively, the biologics is administered concurrently with the compound of Formula I or Formula II. In some embodiments, biologics is administered after the compound of Formula I. In certain embodiments, the biologics is administered after the compound of Formula II.

In some embodiments, the biologics and the compound of Formula I or Formula II are each independently administered by inhalation, intramuscularly, intravenously, intraperitoneally, or subcutaneously. In some embodiments, the biologics and the compound of Formula I or Formula II are each administered intravenously.

In certain embodiments, the method further comprises administering an immune suppressant drug, for example, a corticosteroid. In some embodiments, the immune suppressant drug is selected from tocilizumab, siltuximab, infliximab, abatacept, and anakirna. In certain embodiments, the immune suppressant drug is tocilizumab.

In certain embodiments, the method further comprises administering a vasopressor, such as epinephrine, norepinephrine, phenylephrine, ephedrine, or dopamine.

In certain embodiments, the method further comprises administering a TNF inhibitor, for example, etanercept, adalimumab, certolizumab pegol, golimumab, thalidomide, lenalidomide, pomalidomide, pentoxifylline, or bupropion.

In some embodiments, the method comprises administering the compound of Formula I or Formula II in the form of a pharmaceutical composition that further comprises a pharmaceutically acceptable excipient, or a pharmaceutically acceptable salt thereof.

In some embodiments, the biologics is conjointly administered with the mast cell stabilizer, the compound of Formula III, Formula IV, Formula V, Formula VI, Formula VII, Formula VIII, Formula IX, Formula X, Formula XI, Formula XII, Formula XIII, or Formula XIV, or the anti-inflammatory small molecular peptide truncated from anti-inflammatory gene protein. For example, biologics is administered prior to the mast cell stabilizer, the compound of Formula III-XIV, or the anti-inflammatory small molecular peptide truncated from anti-inflammatory gene protein. Alternatively, the biologics is administered concurrently with the mast cell stabilizer, the compound of Formula III, Formula IV, Formula V, Formula VI, Formula VII, Formula VIII, Formula IX, Formula X, Formula XI, Formula XII, Formula XIII, or Formula XIV, or the anti-inflammatory small molecular peptide truncated from anti-inflammatory gene protein. in certain embodiments, the biologics is administered after the mast cell stabilizer, the compound of Formula III-XIV, or the anti-inflammatory small molecular peptide truncated from anti-inflammatory gene protein. In some embodiments, the biologics and the mast cell stabilizer, the compound of Formula III, Formula IV, Formula V, Formula VI, Formula VII, Formula VIII, Formula IX, Formula X, Formula XI, Formula XII, Formula XIII, or Formula XIV, or the anti-inflammatory small molecular peptide truncated from anti-inflammatory gene protein are each independently administered by inhalation, intramuscularly, intra.venously, intraperitoneally, or subcutaneously.

In certain embodiments, an effective amount of the compound is administered, thereby treating or preventing the condition.

### EXAMPLES

### Example 1

### Cromolvn Treatment Decreases the Levels of Pro-Inflammatory Cytokines in Plasma of TgSOD1 Mice

### Chemicals

Cromolyn. sodium was provide by AZ Therapies and dissolved in PBS. 100 mM solution was used for in vivo experiments. Dulbecco's PBS was used to dilute the solution for intraperitoneal injections for a final dose of 6.3 mg/kg.

### Animals

149 male and female age- and litter-matched transgenic TgSOD1^{G93A} and wild-type WtSOD1^{G93A} mice were used with the following breakdown: Females (19 WtSOD1-Vehicle, 17 WtSOD1-Cromolyn, 19 TgSOD1-Vehicle, and 17 TgSOD1-Cromolyn) and Males (18 WtSOD1-Vehicle, 21 WtSOD1-Cromolyn, 21 TgSOD1-Vehicle, 17 TgSOD1-Cromolyn). The mice received once daily injections of either vehicle or cromolyn sodium (6.3 mg/kg, 96 i.p.) 5 days per week starting at P60 until euthanasia.

All animal care, husbandry and experimentation were performed according to the guidelines set by the Massachusetts General Hospital Subcommittee on Research Animal Care. These experiments were approved by the Massachusetts General Hospital Institutional Animal Care and Use Committee (2014N000018). All mice were given access to food and water ad libitum.

SOD1^{G93A} mice:

B6SJL-Tg (SOD1 G93A)1Gur/J transgenic male mice were obtained from Jackson Laboratory and bred with C57BL/6 female mice to obtain wild-type WtSOD1 and mutant transgenic TgSOD1^{G93A}-expressing mice. To determine mouse genotype, RNA extraction and complimentary DNA (cDNA) synthesis was performed from tail biopsies acquired at postnatal day 28-40 followed by quantitative real-time PCR (qRT-PCR) using primers for the mutant G93A SOD1 gene (GGGAAGCTGTTGTCCCA AG and CAAGGGGAGGTAAAAGAGAGC)(SEQ ID NO: 1 and SEQ ID NO: 2, respectively). Both age- and litter-matched WtSOD1 and TgSOD1 male. and female mice were used for all studies as described below.

### Mesa Scale Discovely Multi-Spot Cytokine Assay

Spinal cord frozen tissue was homogenized in ice-cold RIPA buffer (Thermo Fisher Scientific, #8990) supplemented with protease inhibitor cocktail (Thermo Fisher Scientific, #78430). Samples were centrifuged at 45,000 g for 30 minutes at 4° C. using an Optima TL ultracentrifuge and a TLA 120.2 rotor (Beckman Coulter). Expression levels of the cytokines were assessed in the supernatants derived from spinal cord tissue or in the plasma, using an electrocheiniluminescence-based multi-array method and MESO Quickplex SQ 120 system (MSD, Rockville, Md., USA). The 96-well V-FLEX Proinflammatory Mouse 1 Kit (Meso Scale Discovery, #K15048D) was used to measure simultaneously IL-1β, IL-2, IL-5, IL-6, IL-10, and TNFα, following the manufacturer's instructions. Briefly, samples were diluted in the calibrator and added to the plate coated with an array of cytokine capture antibodies. Samples were incubated. in the plate for 2 hours with shaking at room temperature, followed by washes with the wash buffer provided in the kit. The detection antibody solution was added to each well and the plate was incubated for 2 hours. The plate was washed with the wash buffer and the 2× Read Buffer T was added. The signal was immediately measured on a MESO QuickIlex SQ 120 instrument and was analyzed using the DISCOVERY WORKBENCH 4.0 software (Meso Scale Diagnostics, LLC., Rockville, Md., USA). Protein concentrations in the supernatants or the plasma samples were measured using the Pierce BCA protein assay kit (Thermo Scientific). Values in the graphs represent levels of cytokines normalized to the corresponding protein concentrations.

### Statistics

Data are presented as median values. Box plots are used for graphical representation of population data with the central line representing the median, the edges representing the interquartile ranges, and the whiskers representing 10-90th percentiles. Data are also represented as medians±interquartile ranges or percent values. Sample sizes are included in the figure legends. Comparisons for unrelated samples were performed using a two-way ANOVA followed by Tukey's or Sidak's multiple comparison's test or a one-way ANOVA test followed by Tukey's multiple comparison post-tests at a significance level (a) of 0.05. For p<0.05 and >0.00001, exact P values (two-tailed) are reported.

### Results

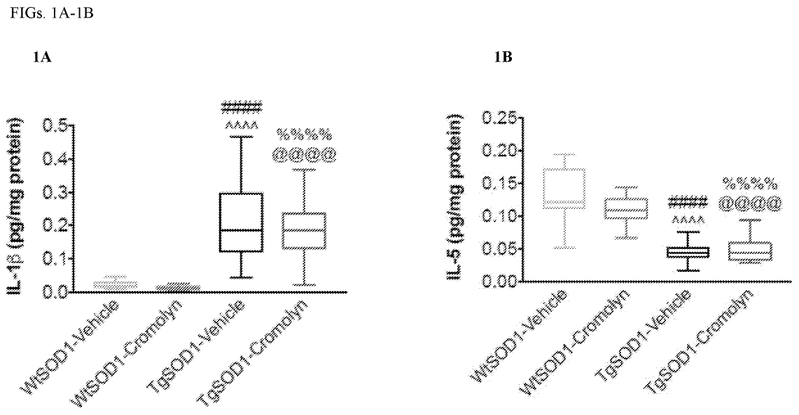

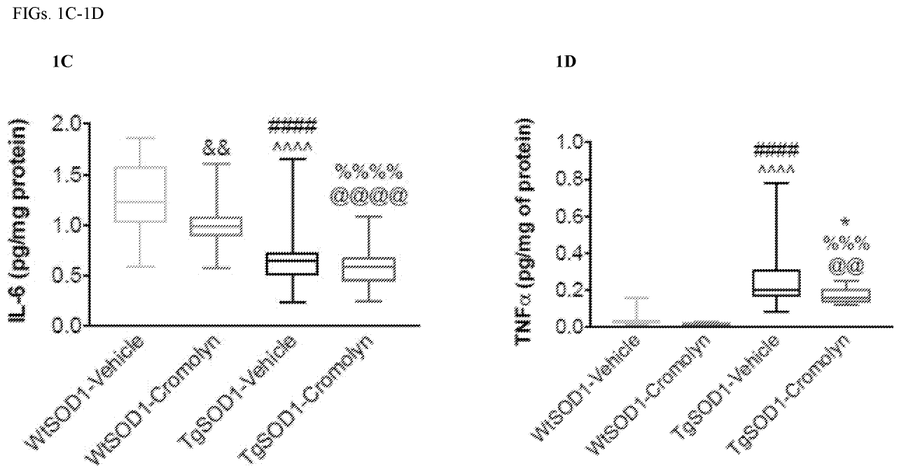

The levels of pro-inflammatory cytokines in spinal cord lysates of mice were measured by using the multi-spot assay system from Meso Scale Discovery. Levels of IL-1β, IL-5, IL-6, IL-10, TNFα have been determined. One-way ANOVA and Tukey's post-hoc analysis revealed a significant difference in the levels of IL-1β [F(3, 130)=66.31, p<0.0001], IL-5 [F(3, 129)=129.9, p<0.0001], IL-6 [F(3, 135)=43.41, p<(10001], and TNFα [F(3, 64)=27.94, p<0.0001], in the spinal cord of both TgSOD1-Vehicle and TgSOD1-Cromolyn groups compared to both wild-type groups (FIGS. 1A, 1B, 1C, and 1D). There was a significant decrease in IL-6 (p<0,0001) and IL-5 (p<0.0001) levels between Tg and Wt groups. Importantly, there was a significant decrease in TNFα (p=0.0273) level in the TgSOD1-Cromolyn group compared to TgSOD1-Vehicle group (FIG. 1D), suggesting that cromolyn treatment decreased expression of pro-inflammatory cytokines and chemokines in the spinal cord of treated transgenic mice.

Cromolyn treatment decreased the levels of pro-inflammatory cytokines in plasma of TgSOD1 mice.

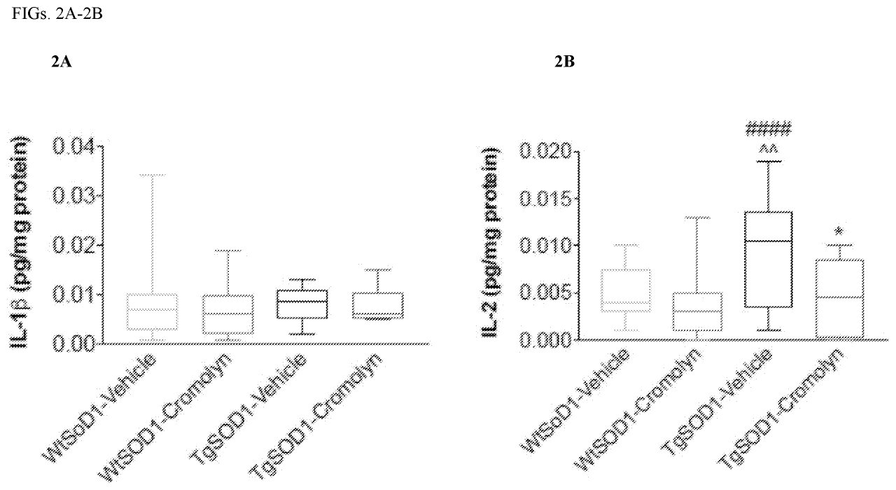

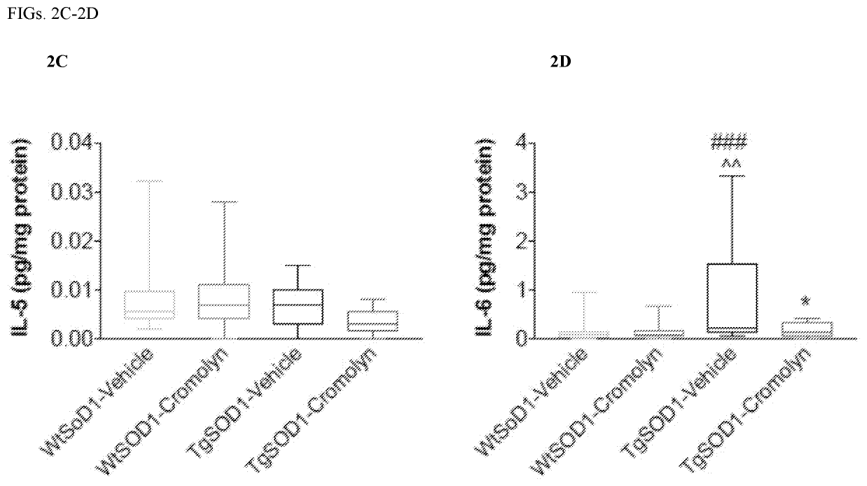

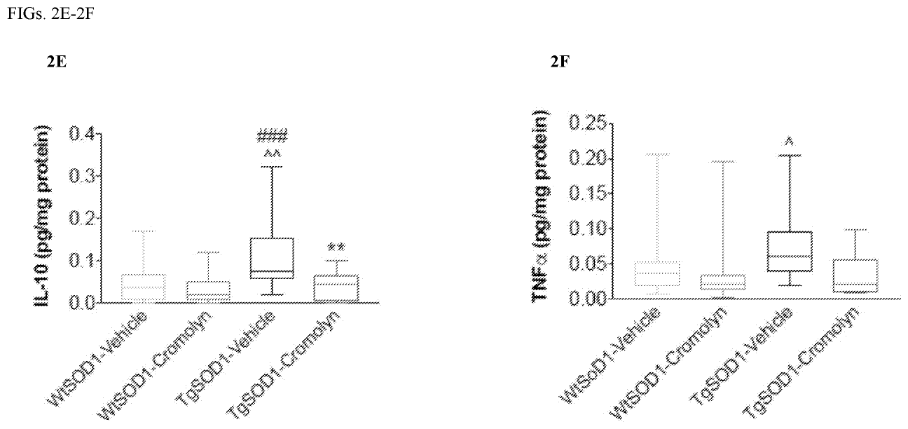

Additionally, levels of L-1β, IL-2, IL-5, IL-6, IL-10, and TNFα were assessed in the plasma of a subset of mice (Females: 13 WtSOD1-Vehicle, 15 WtSOD1-Cromolyn, 6 TgSOD1-Vehicle, and 6 TgSOD1-Cromolyn; and Males: 14 WtSOD1-Vehicle, 10 WtSOD1-Cromolyn, 6 TgSOD1-Vehicle, 3 TgSOD1-Cromolyn). One-way ANOVA and Tukey's post-hoc analysis revealed a significant increase in IL-2 [F(3, 65)=7.731, p<0.0002], IL-6 [F(3, 63)=6.332, p<0.0008], and IL-10 [F(3, 65)=7.195, p<0.0003] levels in the plasma of TgSOD1-Vehicle compared to both WtSOD1-Vehicle and WtSOD1-Cromolyn groups (FIGS. 2B, 2D, and 2E). There was also a significant increase in TNFα levels [F(4, 67)=12.46, p<0.006], and post-hoc analysis revealed a significant increase in TNFα TgSOD1-Vehicle group compared to WISOD1-Cromolyn (p=(10043) (FIG. 2F). There was no statistically significant difference in IL-1β and IL-5 levels between groups (FIGS. 2A and 2C). Importantly, the levels of IL-2 (p =0.0211), IL-6 (p=0.0273), and IL-10 (p=0.0095) were significantly decreased in TgSOD1-Cromolyn group compared to TgSOD1-Vehicle group (FIGS. 2B, 2D, and 2E). Lastly, there was a trend towards a decrease in TNFα levels (p=0.110) in the TgSOD1-Cromolyn mice compared to the TgSOD1-Vehicle group (FIG. 2F). These results demonstrate that cromolyn treatment decreased the levels of cytokines in the plasma of TgSOD1 mice.

### Example 2

### Cromolyn Reverses Pro-Inflammatory CD33-Mediated Inhibition of M1-Microglial Activation Stage in APP/PS1 Mice

Procedure

Naive BV2 microglial cells were treated with DMSO (control) or cromolyn (500 μM) for 16 hours. Afterwards, the cells were incubated with fluoresceraly-labeled A1342 (red) and DMSO or cromolyn for 2 hours. After incubation, the cells were labeled with a plasma membrane dye (PM, green) and imaged. BV2 microglial cells or BV2 cells stably expressing CD33 (BV2-CD33wT) were treated with DMSO or different concentrations of cromolyn for 16 hours. Then, cells were incubated with soluble untagged Aβ42 and DMSO or cromolyn for 2 hours and collected for ELBA analysis. Both naive BV2 and BV2-CD33wT microglial cells treated with cromolyn exhibited increased Aβ42 uptake levels in comparison to cells treated with the vehicle (DMSO).

Results

Interaction of microglia with fibrillar amyloid-1 peptide (Aβ) leads to their phenotypic activation and has recently been suggested to play a role in neuroprotection. It has been shown in numerous studies, in both mice and humans, that glial cells respond to the presence of pathological lesions (plaques and tangles) by changing their morphological characteristics, expressing numerous cell surface receptors, and surrounding the lesions. On the other hand, macrophage and microglial activation in response to cellular debris in the brain, and the subsequent release of pro-flammatory cytokines leads to accelerated neurodegeneration. This, in turn, creates more cellular debris and accelerates disease progression. It is generally agreed that microglia activated by extraceilularly deposited Aβ protect neurons by triggering anti-inflammatory: neurotrophic M2 activation and by clearing Aβ via phagocytosis.

Activation of microglia by extracellularly deposited Aβ is similar to microglial activation in response to the presence of IFNγ, TNFα from T cells, or antigen-presenting cells. Data reveal robust effect of cromolyn in reducing aggregation-prone Aβ levels and inducing a neuroprotective microglial activation state favoring Aβ phagocytosis versus a pro-neuroinflammatory state. This microglial activation is aimed at the protective action in CRS and ICANS. The data obtained for extraceilularly deposited Aβ support the use of cromolyn as a potential drug in the treatment of in CRS and ICANS.

Cromolyn leads to increased recruitment of microglial cells around amyloid plaques, which leads to subsequent Aβ phagocytosis and removal of plaques. Additionally, cromolyn promotes uptake and clearance of Aβ in cultured microglial cells, also leading to removal of plaque.

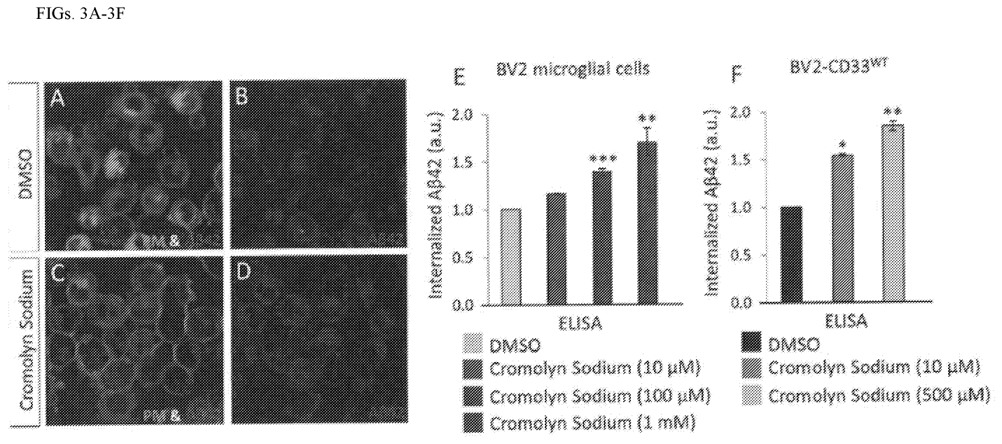

Further, confocal microscopy and enzyme-linked immunosorbent, or ELISA, assays demonstrate the effect of cromolyn on Aβ42 uptake in both BV2 microglial cells and BV2 cells expressing pro-inflammatory human CD33 (BV2-CD33wr), as shown in FIGS. 3A-3D. These data show that cromolyn reverses pro-inflammatory CD33-mediated inhibition of M1-microglial activation stage and leads to increased uptake of Aβ42 in naive BV2 microglial cells. Cromolyn treatment leads to increased Aβ42 uptake in naive BV2 microglial cells as was confirmed by the immunofluorescence results obtained by ELISA (FIG. 3E). Cromolyn leads to increased levels of internalized Aβ42 in BV2-CD33wT cells (FIG. 3F) and reversed CD33-mediated inhibition of Aβ42 uptake in microglial cells. Both naive BV2 and BV2-CD33wT microglial cells treated with cromolyn exhibited increased Aβ42 uptake levels in comparison to cells treated with the vehicle (DMSO). These data demonstrate that treatment with cromolyn shows a dose-dependent effect in modulating Aβ42 uptake levels in naive BV2 and BV2-CD33wT cell lines, thus inhibiting of M1-microglial activation stage, and promoting neuroprotective microglial activation.

### Example 3

### Gene Expression of IL-1β and IL-6 in N9 Microglia Cell Line Stimulated with LPS and Treated with Cromolyn

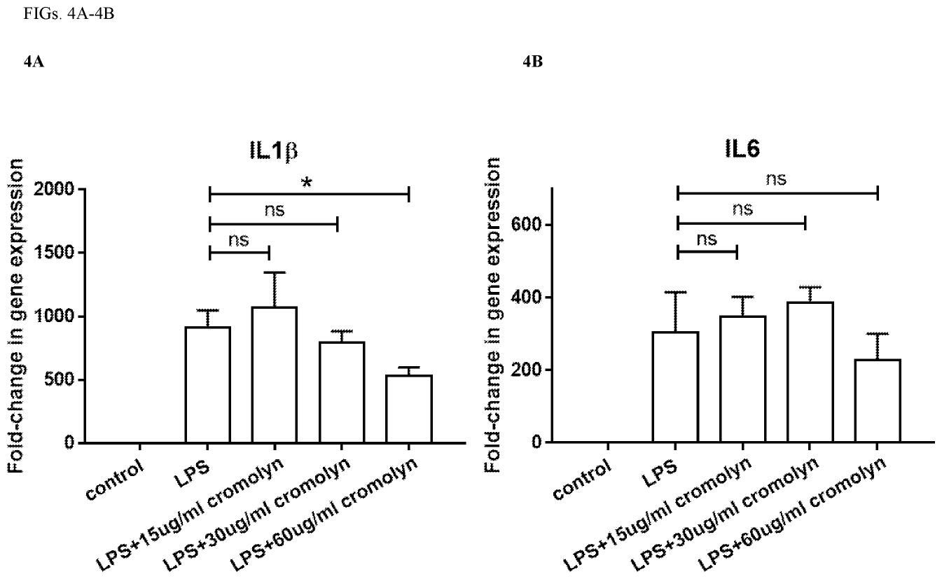

N9 microglia cells were pretreated with different concentrations of cromolyn (15 μg/ml, 30 μg/ml, and 60 μg/ml) for 6 firs and then stimulated with 500 ng/ml lipopolysaccharide (LPS, most commonly used pro-inflammatory stimulus for microglia) in the presence of cromolyn for 8 hrs. Cells was harvested and RNA was isolated with TRUOL (Invitrogen), and first strand cDNA was synthesized using 2 μg of RNA and High-Capacity Reverse Transcriptase (Invitrogen). RT-PCR was performed with SYBR Green PCR reagents on a Bio-Rad detection system. RNA levels were normalized to the level of GAPDH and calculated as delta-delta threshold cycle (ΔΔCT). Primers used for RT-PCR are listed as follows: GAPDH-For: AGCCACATCGCTCAGACAC (SEQ ID NO: 3), GAPDH-Rev: GCCCAATACGACCAAATCC (SEQ ID NO: 4); IL-1β-For; CGCTCAGGGTCACAAGAAAC (SEQ ID NO: 5), IL-1β-Rev: GAGGCAAGGAGGAAAACACA (SEQ ID NO: 6); IL-6-For: TTCCATCCAGTTGCCTTCTT (SEQ ID NO: 7), IL-6-Rev: ATTTCCACGATTTCCCAGAG (SEQ ID NO: 8). Results of the study are shown in FIGS. 4A and 4B.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating at least one condition, wherein the condition is selected from Cytokine Release Syndrome (CRS), Immune effector Cell-Associated Neurotoxicity Syndrome (ICANS), cancer-related cognitive impairment, Infusion Reaction Syndrome (IRS), Capillary Leak Syndrome (CLS), Tumor Lysis Syndrome (TLS), Macrophage Activation Syndrome (MAS), Systemic Inflammatory Response Syndrome (SIRS), Immune Reconstitution Inflammatory Syndrome (IRIS), Graft-Versus-Host Disease (GVHD), Acute Respiratory Distress Syndrome (ARDS), sepsis, Ebola, avian influenza, smallpox, Systemic Inflammatory Response Syndrome (SIRS), and Immune-related Adverse Events Syndrome (IrAES) in a subject in need thereof,
(i) comprising administering a compound of Formula I or Formula II: 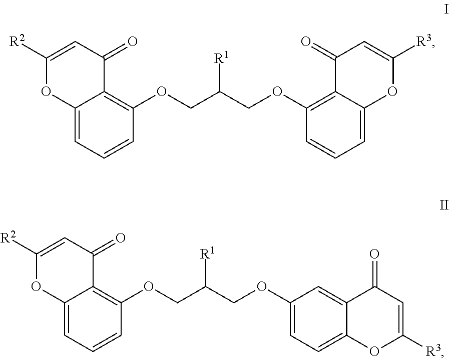
wherein
R¹ is halogen, OH, or —OC(O)C₁₋₅alkyl
R² and R³ are each independently selected from CO₂R⁴ or CH₂OR⁵;
R⁴ is Li, Na, K, H, C₁₋₅alkyl, or —CH₂CO(C₁₋₅alkyl); and
R⁵ is H or —C(O)(C₁₋₅alkyl),
or a pharmaceutically acceptable salt thereof; or
(ii) comprising administering a mast cell stabilizer; or
(iii) comprising administering a compound selected from the compounds of Formula III, Formula IV, Formula V, Formula VI, Formula VII, Formula VIII, Formula IX, Formula X, Formula XI, Formula XII, Formula XIII, and Formula XIV: 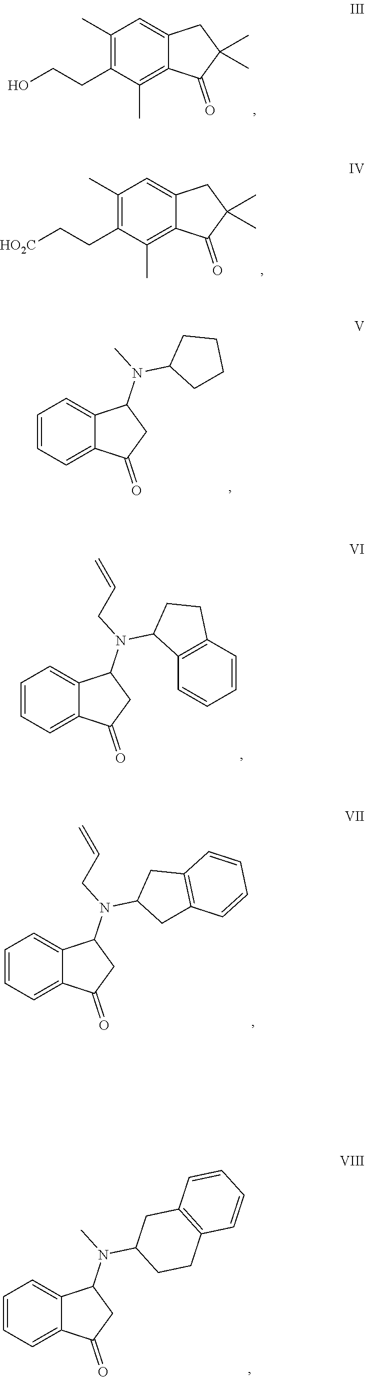 [SMILES: Cc1cc2c(c(C)c1CCO)C(=O)C(C)(C)C2 ; Cc1cc2c(c(C)c1CCC(=O)O)C(=O)C(C)(C)C2 ; CN(C1CCCC1)C1CC(=O)c2ccccc21 ; C=CCN(C1CCc2ccccc21)C1CC(=O)c2ccccc21 ; C=CCN(C1Cc2ccccc2C1)C1CC(=O)c2ccccc21 ; CN(C1CCc2ccccc2C1)C1CC(=O)c2ccccc21] 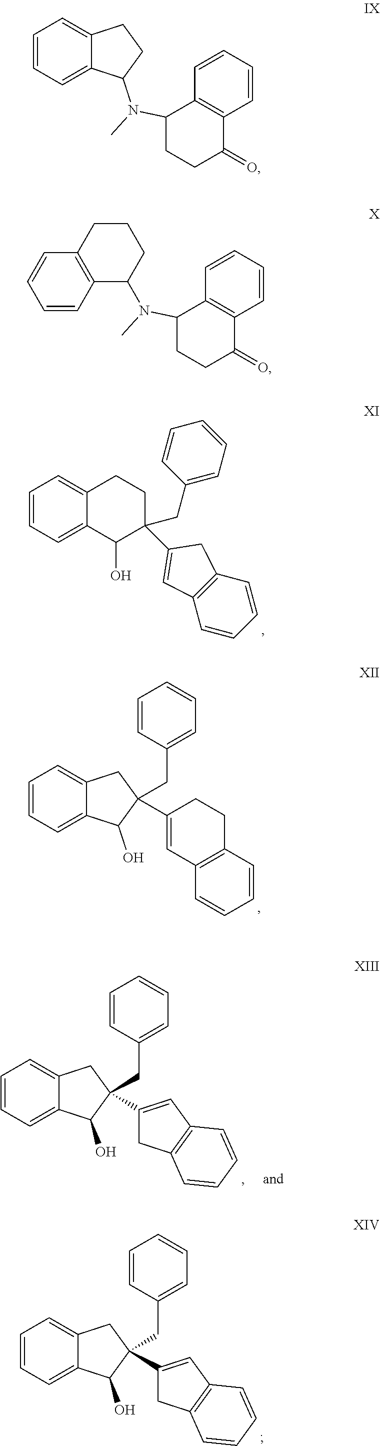 [SMILES: CN(C1CCc2ccccc21)C1CCC(=O)c2ccccc21 ; CN(C1CCCc2ccccc21)C1CCC(=O)c2ccccc21 ; OC1c2ccccc2CCC1(Cc1ccccc1)C1=Cc2ccccc2C1 ; OC1c2ccccc2CC1(Cc1ccccc1)C1=Cc2ccccc2CC1 ; O[C@H]1c2ccccc2C[C@]1(Cc1ccccc1)C1=Cc2ccccc2C1 ; O[C@H]1c2ccccc2C[C@@]1(Cc1ccccc1)C1=Cc2ccccc2C1]
or
(iv) comprising administering an anti-inflammatory small molecular peptide truncated from anti-inflammatory gene protein.

2. The method of claim 71, wherein R¹ is F.

3. The method of claim 71, wherein R¹ is OH.

4-5. (canceled)

6. The method of claim 71, wherein R² and R³ are each independently —CO₂R⁴.

7. The method of claim 71, wherein R⁴ is Li, Na, K, or NH₄.

8. The method of claim 71, wherein R⁴ is Na.

9. The method of claim 71, wherein R⁴ is H.

10. The method of claim 71, wherein R⁴ is C₁₋₅alkyl.

11-14. (canceled)

15. The method of claim 71, wherein the C₁₋₅alkyl is methyl, ethyl, or t-butyl.

16. The method of claim 71, wherein the compound is selected from:

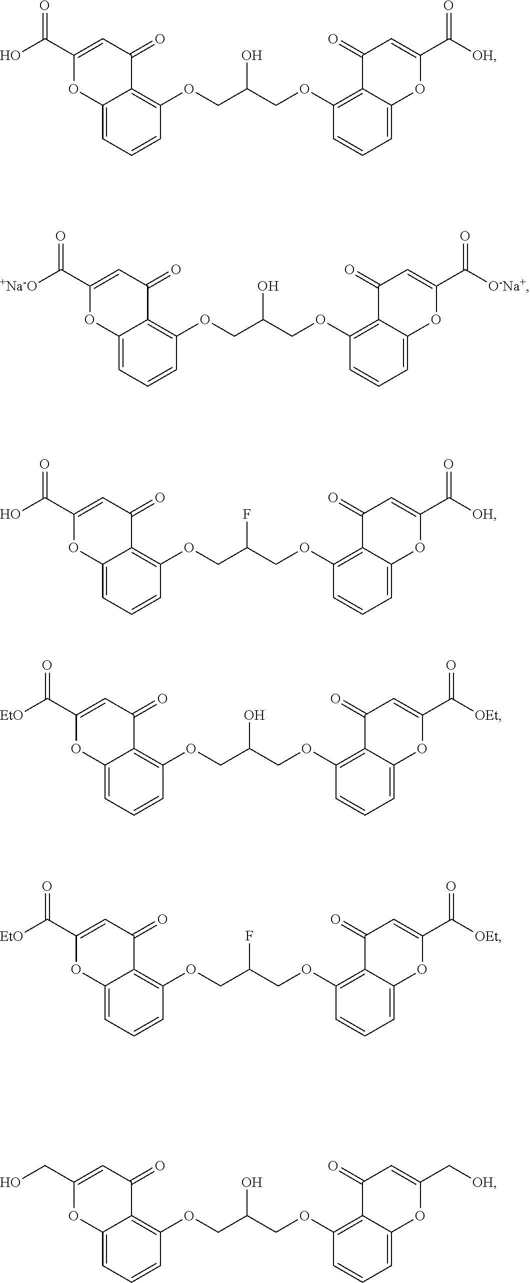

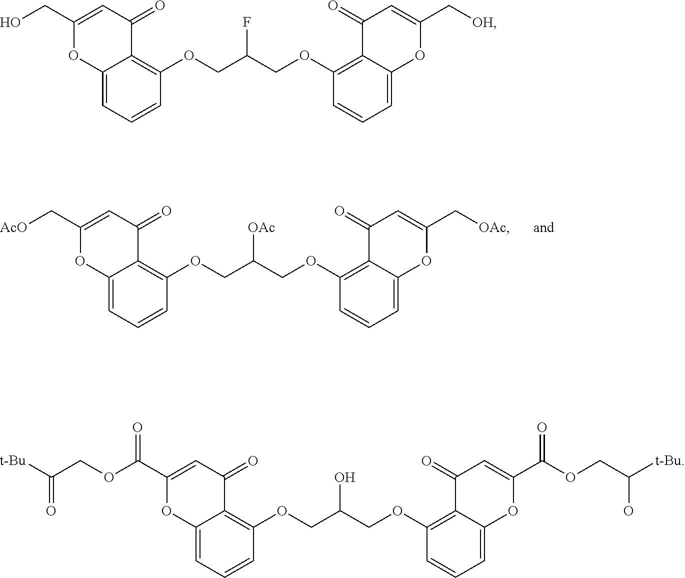 [SMILES: O=c1cc(CO)oc2cccc(OCC(F)COc3cccc4oc(CO)cc(=O)c34)c12 ; CC(=O)OCc1cc(=O)c2c(OCC(COc3cccc4oc(COC(C)=O)cc(=O)c34)OC(C)=O)cccc2o1 ; CC(C)(C)C(=O)COC(=O)c1cc(=O)c2c(OCC(O)COc3cccc4oc(C(=O)OCC(O)C(C)(C)C)cc(=O)c34)cccc2o1]

17-40. (canceled)

41. The method of claim 71, comprising administering the compound in the form of a pharmaceutical composition that further comprises a pharmaceutically acceptable excipient, or a pharmaceutically acceptable salt thereof.

42-70. (canceled)

71. A method of delaying the onset of a disorder or condition in a subject, comprising
administering a compound in an amount sufficient to delay the onset of the disorder or condition in the subject,
wherein the disorder or condition is characterized by aggregation of amyloid plaque, increased macrophage activation, and/or increased microglial activation in the subject; and
wherein the compound has a structure of Formula I or Formula II: 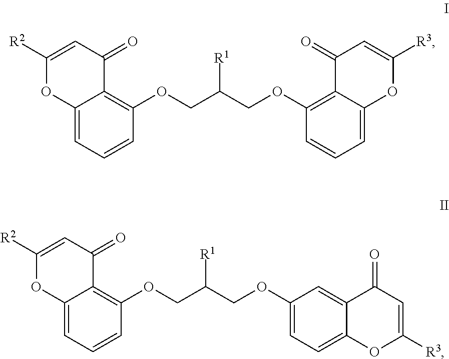
wherein
R¹ is halogen, OH, or —OC(O)C₁₋₅alkyl
R² and R³ are each independently selected from CO₂R⁴ or CH₂OR⁵;
R⁴ is Li, Na, K, H, C₁₋₅alkyl, or —CH₂CO(C₁₋₅alkyl); and
R⁵ is H or C(O)(C₁₋₅alkyl),
or a pharmaceutically acceptable salt thereof

72. The method of claim 71, wherein the compound is

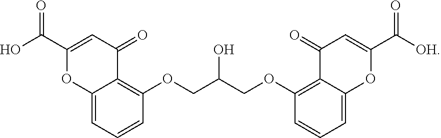 [SMILES: O=C(O)c1cc(=O)c2c(OCC(O)COc3cccc4oc(C(=O)O)cc(=O)c34)cccc2o1]

73. The method of claim 71, wherein the compound is

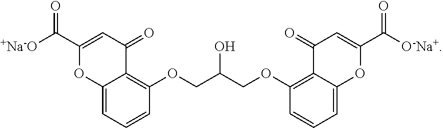

74. The method of claim 71, wherein the compound is

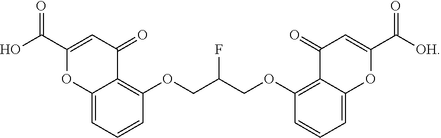 [SMILES: O=C(O)c1cc(=O)c2c(OCC(F)COc3cccc4oc(C(=O)O)cc(=O)c34)cccc2o1]

75. The method of claim 71, wherein the compound is

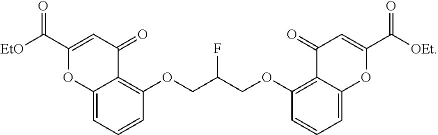 [SMILES: CCOC(=O)c1cc(=O)c2c(OCC(F)COc3cccc4oc(C(=O)OCC)cc(=O)c34)cccc2o1]

76. The method of claim 71, wherein the disease or condition is Alzheimer's disease.
